(19) United States
(12) Patent Application Publication
(10) Pub. No.: US 2018/0044368 A1
(43) Pub. Date: Feb. 15, 2018

(54) GEMCITABINE DERIVATIVES

(71) Applicant: Ligand Pharmaceuticals, Inc., San Diego, CA (US)

(72) Inventor: Lin Zhi, La Jolla, CA (US)

(21) Appl. No.: 15/552,073

(22) PCT Filed: Feb. 23, 2016
(86) PCT No.: PCT/US2016/019176

§ 371 Date: Aug. 18, 2017

Related U.S. Application Data
(60) Provisional application No. 62/120,789, filed on Feb. 25, 2015.
(60) Provisional application No. 62/154,041, filed on Apr. 28, 2015.

(51) Int. Cl. C07H 19/10

(52) U.S. Cl. CPC C07H 19/10

(57) ABSTRACT

Compositions and methods in the field of medicine and chemistry are disclosed. Some of the disclosed embodiments are directed to nucleotide compounds, medicinal compositions, as well as processes for their preparation and methods of their use. In some embodiments, such nucleotide compounds are useful antiviral and antimetabolic agents.

### CROSS REFERENCE TO RELATED APPLICATIONS

This application claims the benefit of U.S. Provisional Application No. 62/154,041 filed Apr. 28, 2015 entitled “NUCLEOTIDE COMPOUNDS AND THEIR USES”, and of U.S. Provisional Application No. 62/120,789 filed Feb. 25, 2015 entitled “NUCLEOTIDE COMPOUNDS AND THEIR USES”, the entire contents of which are incorporated by reference in their entireties.

### FIELD OF THE INVENTION

Compositions and methods in the field of medicine and chemistry are disclosed. Some of the disclosed embodiments are directed to nucleotide compounds, medicinal compositions, as well as processes for their preparation and methods of their use. In some embodiments, such nucleotide compounds are useful antiviral and antimetabolic agents.

### BACKGROUND OF THE INVENTION

The following description of the background is provided to aid in understanding the invention, but is not admitted to be, or to describe, prior art to the invention.

Nucleoside/nucleotide and their derivative compounds have been widely used as antiviral and antimetabolic therapeutic agents to treat viral diseases such as HIV, HCV, and HBV, and solid tumors. Despite of significant advances in the field of new generation of nucleos(t)ide based drugs with improved therapeutic activity and pharmacokinetic properties, new agents are still needed to combat drug resistance and genetic heterogeneity.

### SUMMARY OF THE INVENTION

Novel nucleoside and nucleotide compounds of gemcitabine derivatives such as phosphates, and phosphoramidates, their preparation and their uses are described. Some embodiments are related to the use of prodrugs to treat viral diseases and various carcinomas, including but not limited to hepatitis, liver fibrosis, fatty liver, malaria, HIV, HPV, non-small cell lung cancer, pancreatic cancer, bladder cancer, breast cancer, liver cancer, lymphomas, and other types of tumors.

Some embodiments relate to a compound of Formula I:

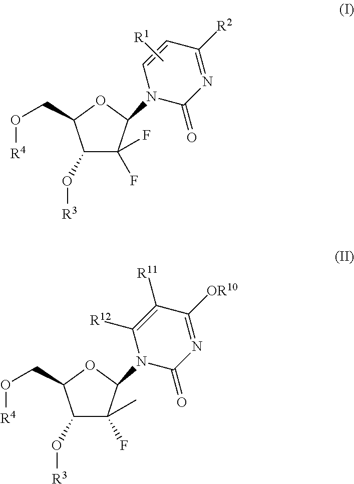

wherein:
R¹ is a halogen, hydrogen isotope, or an optionally substituted C₁-C₆ alkyl;
R² is selected from the group consisting of OR⁵ and NR⁵R⁶;
R³ is selected from the group consisting of H, an optionally substituted C₁-C₆ alkyl, an optionally substituted C₁-C₆ hetereoalkyl, and an optionally substituted C₁-C₆ alkylacyl;
R⁴ is selected from the group consisting of H, monophosphate, diphosphate, triphosphate, and

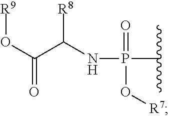

or R³ and R⁴ can be optionally linked;
R⁵ is selected from the group consisting of H, deuterium, an optionally substituted C₁-C₈ alkyl, an optionally substituted C₁-C₈ alkylacyl, an optionally substituted arylacyl, and an optionally substituted heteroarylacyl;
R⁶ is selected from the group consisting of deuterium, an optionally substituted C₁-C₈ alkylacyl, an optionally substituted arylacyl, and an optionally substituted heteroarylacyl; or R⁶ is H when R¹ is deuterium;
R⁷ is a phenyl or naphthalenyl;
R⁸ and R⁹ are independently a C₁-C₆ alkyl;
R¹⁰ is selected from the group consisting of H, an optionally substituted C₁-C₈ alkyl, an optionally substituted C₁-C₈ alkylacyl, an optionally substituted arylacyl, and an optionally substituted heteroarylacyl;
R¹¹ and R¹² are deuterium; or R¹¹ is deuterium and R¹² is hydrogen; or R¹² is deuterium and R¹¹ is hydrogen;
or a stereoisomer or a tautomer or a pharmaceutically acceptable salt thereof.

In some embodiments, the compound is selected from the group consisting

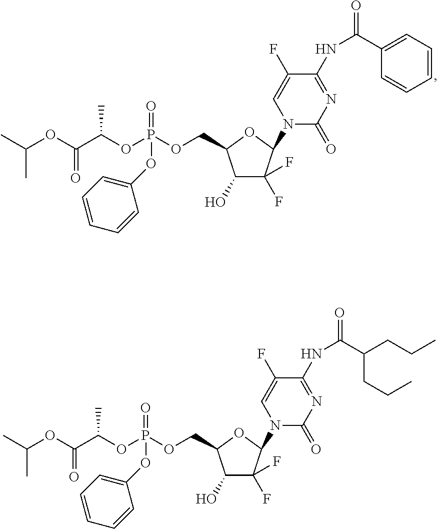 [SMILES: CC(C)OC(=O)[C@H](C)OP(=O)(OC[C@H]1O[C@@H](n2cc(F)c(NC(=O)c3ccccc3)nc2=O)C(F)(F)[C@@H]1O)Oc1ccccc1 ; CCCC(CCC)C(=O)Nc1nc(=O)n([C@@H]2O[C@H](COP(=O)(Oc3ccccc3)O[C@@H](C)C(=O)OC(C)C)[C@@H](O)C2(F)F)cc1F]

and a stereoisomer or a pharmaceutically acceptable salt thereof.

Some embodiments relate to a method of treating a disease, disorder or condition comprising administering an effective amount of any of the above compounds to a subject in need thereof.

In some embodiments, the disease is a viral disease.

In some embodiments, the disease is certain types of cancer.

Some embodiments further comprise administering an effective amount of at least one additional therapeutic agent to the subject in need thereof.

In some embodiments, the subject is a mammal.

In some embodiments, the subject is human.

Some embodiments relate to a method of inhibiting viral or tumor replication in a cell comprising contacting the cell with any of the above compounds.

In some embodiments, the cell is in vivo.

In some embodiments, the cell is ex vivo.

In some embodiments, the cell is mammalian.

In some embodiments, the cell is human.

### DETAILED DESCRIPTION

The present embodiments are directed to compositions and methods related to novel compounds of biologically active nucleoside derivatives such as nucleoside phosphates, nucleoside phosphonamidates, their preparation and their uses. These compounds and their stereoisomers and pharmaceutically acceptable salts are represented by Formula I:

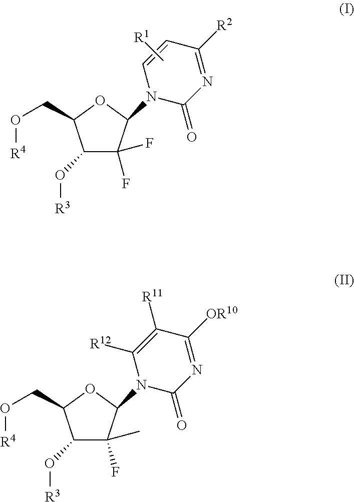

wherein:
R¹ is a halogen, hydrogen isotope, or an optionally substituted C₁-C₆ alkyl;
R² is selected from the group consisting of OR⁵ and NR⁵R⁶;
R³ is selected from the group consisting of H, an optionally substituted C₁-C₆ alkyl, an optionally substituted C₁-C₆ hetereoalkyl, and an optionally substituted C₁-C₆ alkylacyl;
R⁴ is selected from the group consisting of H, monophosphate, diphosphate, triphosphate, and

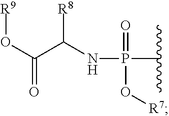

or R³ and R⁴ can be optionally linked;
R⁵ is selected from the group consisting of H, deuterium, an optionally substituted C₁-C₈ alkyl, an optionally substituted C₁-C₈ alkylacyl, an optionally substituted arylacyl, and an optionally substituted heteroarylacyl;
R⁶ is selected from the group consisting of deuterium, an optionally substituted C₁-C₈ alkylacyl, an optionally substituted arylacyl, and an optionally substituted heteroarylacyl; or R⁶ is H when R¹ is deuterium;
R⁷ is a phenyl or naphthalenyl;
R⁸ and R⁹ are independently a C₁-C₆ alkyl;
R¹⁰ is selected from the group consisting of H, an optionally substituted C₁-C₈ alkyl, an optionally substituted C₁-C₈ alkylacyl, an optionally substituted arylacyl, and an optionally substituted heteroarylacyl;
R¹¹ and R¹² are deuterium; or R¹¹ is deuterium and R¹² is hydrogen; or R¹² is deuterium and R¹¹ is hydrogen;
or a stereoisomer or a tautomer or a pharmaceutically acceptable salt thereof.

Certain drugs of phosphate derivatives are highly charged compounds that have generally poor oral bioavailability due to poor absorption in the gastrointestinal tract. Certain drugs are highly lipophilic compounds that have generally poor oral bioavailability due to poor absorption in the gastrointestinal tract. In some embodiments, the compounds of Formula I have oral bioavailability superior to the parent drugs/agents.

Compounds of Formula I have asymmetric centers where the stereochemistry is unspecified, and the diastereomeric mixtures of these compounds are included, as well as the individual stereoisomers when referring to a compound of Formula I generally.

Certain compounds of Formula I contains a heterocycle where the double bonds can be tautomerized to a different configuration, and the tautomeric mixtures of these compounds are included, as well as the individual tautomers when referring to a compound of Formula I generally.

Some embodiments of the compounds, compositions and methods provided herein include a pharmaceutical composition comprising a compound provided herein and a pharmaceutically acceptable carrier.

Some embodiments also include administering an effective amount of a second or multiple therapeutic agents in combination with a compound provided herein to the subject in need thereof.

Some embodiments of the compounds, compositions and methods provided herein include a method of treating a viral disease comprising administering an effective amount of a compound provided herein to a subject in need thereof.

Some embodiments of the compounds, compositions and methods provided herein include a method of treating various types of cancers comprising administering an effective amount of a compound provided herein to a subject in need thereof.

In some embodiments, the subject is mammalian.

In some embodiments, the subject is human.

Some embodiments of the compounds, compositions and methods provided herein include a method of testing a compound in a cell comprising contacting the cell with the compound of the claims.

In some embodiments, the cell is in vivo.

In some embodiments, the cell is ex vivo.

In some embodiments, the cell is mammalian.

In some embodiments, the cell is human.

### Definitions

In accordance with the present disclosure and as used herein, the following terms are defined with the following meanings, unless explicitly stated otherwise. It is to be understood that both the foregoing general description and the following detailed description are exemplary and explanatory only and are not restrictive of the subject matter claimed. In this application, the use of the singular includes the plural unless specifically stated otherwise. In this application, the use of “or” means “and/or” unless stated otherwise. Furthermore, use of the term “including” as well as other forms, such as “includes,” and “included” is not limiting.

As used herein, ranges and amounts can be expressed as “about” a particular value or range. “About” also includes the exact amount. Hence “about 10%” means “about 10%” and also “10%.”

As used herein, “optional” or “optionally” means that the subsequently described event or circumstance does or does not occur, and that the description includes instances where the event or circumstance occurs and instances where it does not. For example, an optionally substituted group means that the group is unsubstituted or is substituted.

As used herein, the singular forms “a,” “an” and “the” include plural referents unless the context clearly dictates otherwise. Thus, for example, reference to a composition comprising “a therapeutic agent” includes compositions with one or a plurality of therapeutic agents.

The term “alkyl” refers to saturated aliphatic groups including straight-chain, branched chain and cyclic groups, up to and including 10 carbon atoms. Suitable alkyl groups include methyl, ethyl, n-propyl, isopropyl, and cyclopropyl. The alkyl group may be optionally substituted with 1-3 substituents.

The term “optionally substituted” or “substituted” includes groups substituted by one to four substituents, independently selected from lower alkyl, lower aryl, lower aralkyl, lower cyclic alkyl, lower heterocycloalkyl, hydroxy, lower alkoxy, lower aryloxy, perhaloalkoxy, aralkoxy, lower heteroaryl, lower heteroaryloxy, lower heteroarylalkyl, lower heteroaralkoxy, azido, amino, halogen, lower alkylthio, oxo, lower acylalkyl, lower carboxy esters, carboxyl, carboxamido, nitro, lower acyloxy, lower aminoalkyl, lower alkylaminoaryl, lower alkylaryl, lower alkylaminoalkyl, lower alkoxyaryl, lower arylamino, lower aralkylamino, lower alkylsulfonyl, lower carboxamidoalkylaryl, lower carboxamidoaryl, lower hydroxyalkyl, lower haloalkyl, lower alkylaminoalkylcarboxy, lower aminocarboxamidoalkyl, cyano, lower alkoxyalkyl, lower perhaloalkyl, phosphate, phosphonate, or phosphoramidate, and lower arylalkyloxyalkyl. “Substituted aryl” and “substituted heteroaryl” refers to aryl and heteroaryl groups substituted with 1-6 substituents. These substituents are selected from the group consisting of lower alkyl, lower alkoxy, lower perhaloalkyl, halogen, hydroxy, cyano, and amino.

The term “heteroalkyl” refer to alkyl groups containing at least one heteroatom, such as 1 to 3 heteroatoms. Suitable heteroatoms include oxygen, sulfur, and nitrogen.

The term “heteroalkylacyl” refer to —C(O)-heteroalkyl groups.

The term “alkylacyl” refer to —C(O)-alkyl groups.

The term “acyloxy” refers to —OC(O)R where R is alkyl, or heteroalkyl.

The term “alkoxy” or “alkyloxy” refers to OR where R is alkyl, or heteroalkyl, all optionally substituted.

The term “carboxyl” refers to C(O)OH.

The term “oxo” refers to an ═O group.

The term “amino” refers to NRR′ where R and R′ are each independently selected from hydrogen, alkyl, aryl, aralkyl and heterocycloalkyl, all except H are optionally substituted; and R and R′ can form a cyclic ring system.

The term ‘acylamino” refers to —NRC(O)R′ where R and R′ are each independently selected from H, alkyl, or heteroalkyl.

The term “halogen” or “halo” refers to F, Cl, Br and I.

The term “haloalkyl” refer to alkyl groups containing at least one halogen, in a further aspect are 1 to 3 haloatoms. Suitable haloatoms include F, Cl, and Br.

The term “haloacyl” refer to —C(O)-haloalkyl groups.

The term “alkenyl” refers to unsaturated groups which have 2 to 12 atoms and contain at least one carbon carbon double bond and includes straight chain, branched chain and cyclic groups. Alkenyl groups may be optionally substituted. Suitable alkenyl groups include allyl.

The term “alkynyl” refers to unsaturated groups which have 2 to 12 atoms and contain at least one carbon carbon triple bond and includes straight chain, branched chain and cyclic groups. Alkynyl groups may be optionally substituted. Suitable alkynyl groups include ethynyl.

The term “aminoalkyl” refers to the group NR₂-alkyl where R is selected from H, alkyl, aryl, aralkyl, and heterocycloalkyl.

The terms “alkylthio” refers to the group alkyl-S—.

The term “amido” refers to the NR₂ group next to an acyl or sulfonyl group as in NR₂C(O)—, RC(O)NR—, NR₂S(═O)₂— and RS(═O)₂—NR—, where R includes H, alkyl, aryl, aralkyl, and heterocycloalkyl.

The term “perhalo” refers to groups wherein every C—H bond has been replaced with a C-halo bond on an aliphatic or aryl group. Suitable perhaloalkyl groups include CF₃ and CFCl₂.

The term “aryl” refers to an aromatic group wherein each of the atoms forming the ring is a carbon atom. Aryl rings may be formed by five, six, seven, eight, nine, or more than nine carbon atoms. Aryl groups may be optionally substituted. Examples of aryl groups include, but are not limited to phenyl, naphthalenyl, phenanthrenyl, anthracenyl, tetralinyl, fluorenyl, indenyl, and indanyl.

The term “heteroaryl” refers to an aromatic group wherein at least one atom forming the aromatic ring is a heteroatom. Heteroaryl rings may be formed by three, four, five, six, seven, eight, nine, or more than nine atoms. Heteroaryl groups may be optionally substituted. Examples of heteroaryl groups include, but are not limited to, aromatic C₃₋₈ heterocyclic groups comprising one oxygen or sulfur atom or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In some embodiments, heteroaryl groups are optionally substituted with one or more substituents, independently selected from halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline. In some embodiments, the substituents are halo, hydroxy, cyano, O—C₁₋₆-alkyl, C₁₋₆-alkyl, hydroxy-C₁₋₆-alkyl, and amino-C₁₋₆-alkyl.

The term “arylacyl” refers to —C(O)-aryl groups.

The term “heteroarylacyl” refers to —C(O)-heteroaryl groups.

The phrase “therapeutically effective amount” means an amount of a compound or a combination of compounds that partially or fully ameliorates, attenuates or eliminates one or more of the symptoms of a particular disease or condition or prevents, modifies, or delays the onset of one or more of the symptoms of a particular disease or condition. Such amount can be administered as a single dosage or can be administered according to a regimen, whereby it is effective. Repeated administration may be needed to achieve a desired result (e.g., treatment of the disease and/or condition).

The term “pharmaceutically acceptable salt” includes salts of compounds of Formula I and its prodrugs derived from the combination of a compound of the present embodiments and an organic or inorganic acid or base. Suitable acids include acetic acid, adipic acid, benzenesulfonic acid, (+)-7,7-dimethyl-2-oxobicyclo[2.2.1]heptane-1-methanesulfonic acid, citric acid, 1,2-ethanedisulfonic acid, dodecyl sulfonic acid, fumaric acid, glucoheptonic acid, gluconic acid, glucuronic acid, hippuric acid, hydrochloride hemiethanolic acid, HBr, HCl, HI, 2-hydroxyethanesulfonic acid, lactic acid, lactobionic acid, maleic acid, methanesulfonic acid, methylbromide acid, methyl sulfuric acid, 2-naphthalenesulfonic acid, nitric acid, oleic acid, 4,4′-methylenebis-[3-hydroxy-2-naphthalenecarboxylic acid], phosphoric acid, polygalacturonic acid, stearic acid, succinic acid, sulfuric acid, sulfosalicylic acid, tannic acid, tartaric acid, terphthalic acid, and p-toluenesulfonic acid.

Where the number of any given substituent is not specified (e.g., “haloalkyl”), there may be one or more substituents present. For example, “haloalkyl” can include one or more of the same or different halogens. For example, “haloalkyl” includes each of the substituents CF₃, CHF₂ and CH₂F.

The term “patient” refers to an animal being treated including a mammal, such as a dog, a cat, a cow, a horse, a sheep, and a human. In some embodiments the patient is a mammal, either male or female. In some embodiments, the patient is a male or female human.

The term “prodrug” as used herein refers to any compound that when administered to a biological system generates a biologically active compound as a result of spontaneous chemical reaction(s), enzyme catalyzed chemical reaction(s), and/or metabolic chemical reaction(s), or a combination of each. Standard prodrugs are formed using groups attached to functionality, e.g. HO—, HS—, HOOC—, HOOPR₂—, associated with the drug, that cleave in vivo. Standard prodrugs include but are not limited to carboxylate esters where the group is alkyl, aryl, aralkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. The groups illustrated are examples, not exhaustive, and one skilled in the art could prepare other known varieties of prodrugs. Such prodrugs of the compounds of Formula I fall within this scope. Prodrugs must undergo some form of a chemical transformation to produce the compound that is biologically active or is a precursor of the biologically active compound. In some cases, the prodrug is biologically active, usually less than the drug itself, and serves to improve drug efficacy or safety through improved oral bioavailability, pharmacodynamic half-life, etc. Prodrug forms of compounds may be utilized, for example, to improve bioavailability, improve subject acceptability such as by masking or reducing unpleasant characteristics such as bitter taste or gastrointestinal irritability, alter solubility such as for intravenous use, provide for prolonged or sustained release or delivery, improve ease of formulation, or provide site specific delivery of the compound.

The term “stereoisomer” refers to the relative or absolute spatial relationship of the R⁸ group and the nucleoside attached to the phosphorus atom via a single bond, and refers to individual or any combination of the individual isomers such as a racemic mixture and a diastereomeric mixture. For example, the structures A, B, C, and D below show four possible individual isomers. Structures A and D (or B and C) are a pair of two enantiomers (or called optical isomers).

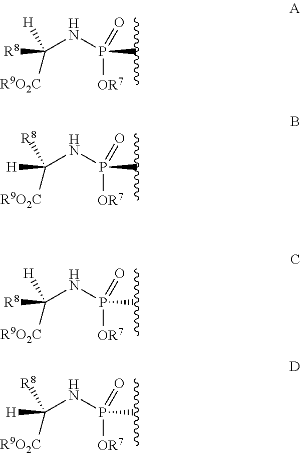

The term “tautomer” refers to the isomers whose double bonds can exist in more than one configuration. For example, base moiety of the compounds of Formula I can exist as tautomeric forms as below.

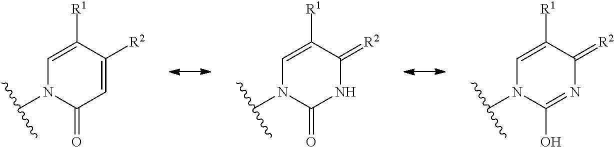

The terms “treating” or “treatment” of a disease includes inhibiting the disease (slowing or arresting or partially arresting its development), providing relief from the symptoms or side effects of the disease (including palliative treatment), and/or relieving the disease (causing regression of the disease).

The terms “biological agent” refers to a compound that has biological activity or that has molecular properties that can be used for diagnosis purpose, such as a compound carrying a radioactive isotope or a heavy atom.

### Formulations

The disclosed compounds may be used alone or in combination with other treatments. These compounds, when used in combination with other agents, may be administered as a daily dose or an appropriate fraction of the daily dose (e.g., bid). The compounds may be administered after a course of treatment by another agent, during a course of therapy with another agent, administered as part of a therapeutic regimen, or may be administered prior to therapy by another agent in a treatment program.

Examples of pharmaceutically acceptable salts include acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulfate, napsylate, nitrate, oleate, palmoate, phosphate, polygalacturonate, stearate, succinate, sulfate, sulfosalicylate, tannate, tartrate, terphthalate, tosylate, and triethiodide.

Compositions containing the active ingredient may be in any form suitable for the intended method of administration. In some embodiments, the compounds of a method and/or composition described herein can be provided via oral administration, rectal administration, transmucosal administration, intestinal administration, enteral administration, topical administration, transdermal administration, intrathecal administration, intravenous administration, intramuscular administration, intraventricular administration, intraperitoneal administration, intranasal administration, intraocular administration and/or parenteral administration.

When the compounds are administered via oral administration, for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient can be mixed with an inert solid diluent, for example calcium phosphate or kaolin, or as soft gelatin capsules wherein the active ingredient can be mixed with water or an oil medium, such as peanut oil, liquid paraffin or olive oil.

Formulations suitable for parenteral administration include aqueous and non-aqueous isotonic sterile injection solutions which may contain, for example, antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

In some embodiments unit dosage formulations contain a daily dose or unit, daily sub-dose, or an appropriate fraction thereof, of a drug. It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

### Synthesis of Compounds

The following procedures for the preparation of the compounds illustrate the general procedures used to prepare the compounds. Optically pure prodrugs containing a single isomer at the phosphorus center can be made, for example, by separation of the diastereomers by a combination of column chromatography and/or crytallyzation, or by enantioselective synthesis of chiral activated phosphate intermediates.

Scheme I describes general strategies of derivatizing the 3-hydroxy group of the compounds of Formula I. The first strategy starts with protection of the primary hydroxy group of compounds of structure 1 with a standard protection groups such as alkylsilyl or acyl to give compounds of structure 2. Akylation or acylation followed by deprotection afford the nucleoside product of structure 3.

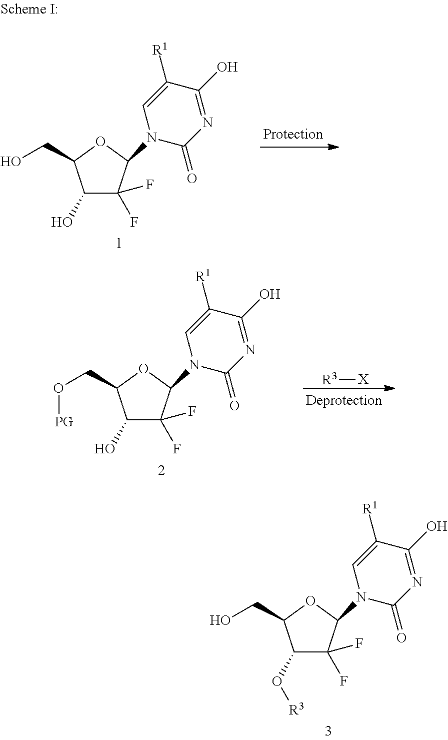

Scheme II describes general strategies of derivatizing the 4′-amino group of compounds of Formula I. Protection of the dihydroxy groups of compounds of structure 4 with a standard protection group such as alkylsilyl or acyl provides compounds of structure 5. Acylation of the animo group with a standard acylating agent such as acid chloride, anhydride, or carbamide followed by de-protection yields product of structure 6.

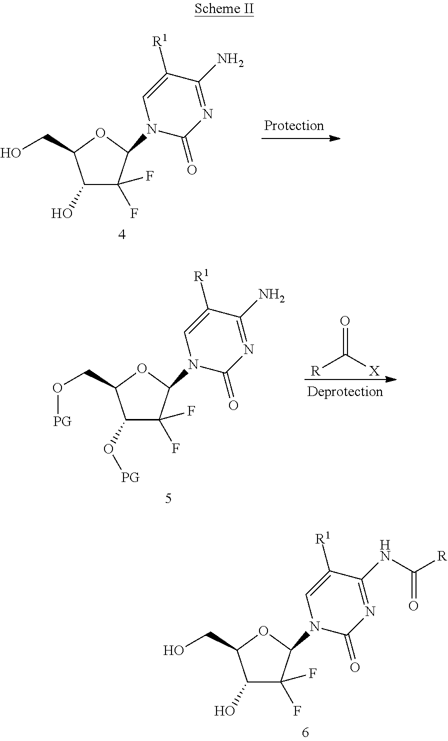

Scheme III describes general synthetic procedures of the phosphoramidate prodrugs of Formula I. A nucleoside derivative of structure 7 is treated with the phosphorus chloride of structure to provide final product of structure 9.

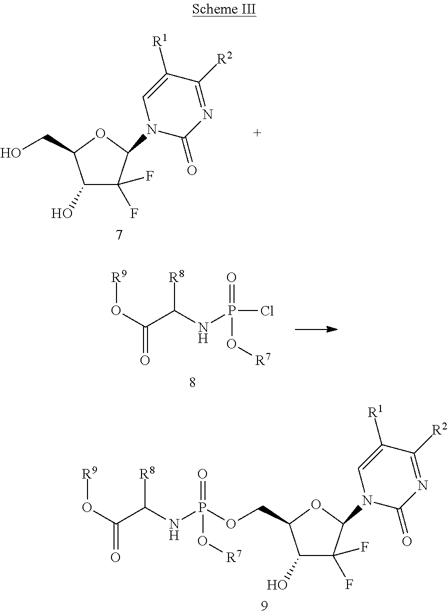

### EXAMPLES

Some biologically active compounds of Formula I are prepared as outlined below.

### Example 1

### Isopropyl (2S)-2-(((((2R,3R,5R)-5-(4-benzamido-5-fluoro-2-oxopyrimidin-1(2H)-yl)-4,4-difluoro-3-hydroxytetrahydrofuran-2-yl)methoxy)(phenoxy)phosphoryl)oxy)propanoate (Compound 101)

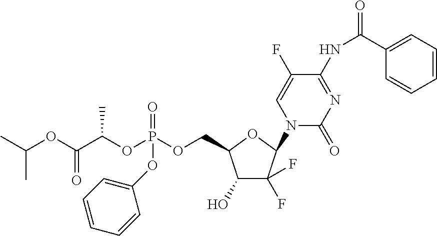 [SMILES: CC(C)OC(=O)[C@H](C)OP(=O)(OC[C@H]1O[C@@H](n2cc(F)c(NC(=O)c3ccccc3)nc2=O)C(F)(F)[C@@H]1O)Oc1ccccc1]

Compound 101 is prepared as a mixture of two diastereomers according to synthetic strategy of Scheme I-III from 5′-fluoro-gemcitabine. [M+H]⁺ calcd for C₂₈H₂₉F₃N₃O₁₀P: 655.15.

### Example 2

### Isopropyl (2S)-2-(((((2R,3R,5R)-4,4-difluoro-5-(5-fluoro-2-oxo-4-(2-propylpentanamido)pyrimidin-1 (2H)-yl)-3-hydroxytetrahydrofuran-2-yl)methoxy)(phenoxy)phosphoryl)oxy)propanoate

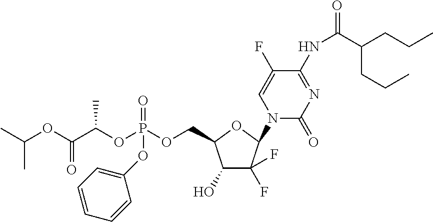 [SMILES: CCCC(CCC)C(=O)Nc1nc(=O)n([C@@H]2O[C@H](COP(=O)(Oc3ccccc3)O[C@@H](C)C(=O)OC(C)C)[C@@H](O)C2(F)F)cc1F]

Compound 102 is prepared as a mixture of two diastereomers according to synthetic strategy of Scheme I-III from 5′-fluoro-gemcitabine. [M+H]⁺ calcd for C₂₉H₃₉F₃N₃O₁₀P: 677.23.

### Biological Examples

Examples of use of the method include the following. It will be understood that the following are examples and that the method is not limited solely to these examples.

### Example A: In Vitro Antiviral Activity Assay

Antiviral activity of the novel nucleosides are tested in triphosphate forms in a standard assay against a purified viral polymerase such as HCV or HBV.

### Methods:

The testing nucleoside triphosphate is prepared from the corresponding nucleoside by standard nucleotide synthesis.

### Example B: In Vitro Antiproliferation Activity Assay

Antitumor activity of the novel compounds are tested in a variety of cancer cell lines against reference compound gemicitabine.

### Example C: Tissue Distribution Following Oral Administration of Active Compounds and their Prodrugs

Efficiency to generate the nucleoside triphosphate in specified tissues after oral administration is measured in the rats.

### Methods:

Nucleoside analogues and their prodrugs are administered at 5-20 mg/kg to fasted rats by oral gavage. Plasma and portal vein concentrations of the active and prodrug are determined by HPLC-UV, and the liver, skeletal muscle, cardiac, kidney, small intestine, and other organ concentrations are measured by LC-MS using the standard chromatography method. The results demonstrate the liver targeting of the prodrug compounds and provide evidence for improved safety of the prodrugs over that of the actives.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A compound of Formula I: 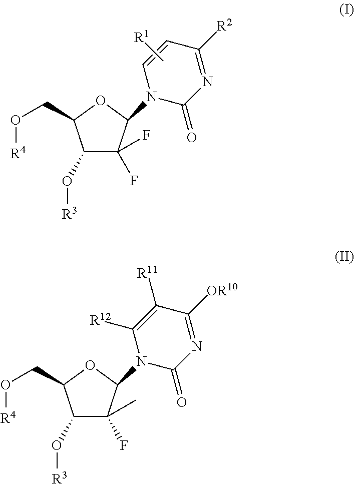
wherein:
R¹ is a halogen, hydrogen isotope, or an optionally substituted C₁-C₆ alkyl;
R² is selected from the group consisting of OR⁵ and NR⁵R⁶;
R³ is selected from the group consisting of H, an optionally substituted C₁-C₆ alkyl, an optionally substituted C₁-C₆ hetereoalkyl, and an optionally substituted C₁-C₆ alkylacyl;
R⁴ is selected from the group consisting of H, monophosphate, diphosphate, triphosphate, and 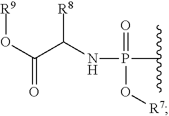
or R³ and R⁴ can be optionally linked;
R⁵ is selected from the group consisting of H, deuterium, an optionally substituted C₁-C₈ alkyl, an optionally substituted C₁-C₈ alkylacyl, an optionally substituted arylacyl, and an optionally substituted heteroarylacyl;
R⁶ is selected from the group consisting of deuterium, an optionally substituted C₁-C₈ alkylacyl, an optionally substituted arylacyl, and an optionally substituted heteroarylacyl; or R⁶ is H when R¹ is deuterium;
R⁷ is a phenyl or naphthalenyl;
R⁸ and R⁹ are independently a C₁-C₆ alkyl;
R¹⁰ is selected from the group consisting of H, an optionally substituted C₁-C₈ alkyl, an optionally substituted C₁-C₈ alkylacyl, an optionally substituted arylacyl, and an optionally substituted heteroarylacyl;
R¹¹ and R¹² are deuterium; or R¹¹ is deuterium and R¹² is hydrogen; or R¹² is deuterium and R¹¹ is hydrogen;
or a stereoisomer or a tautomer or a pharmaceutically acceptable salt thereof.

2. The compound of claim 1, wherein the compound is selected from the group consisting of: 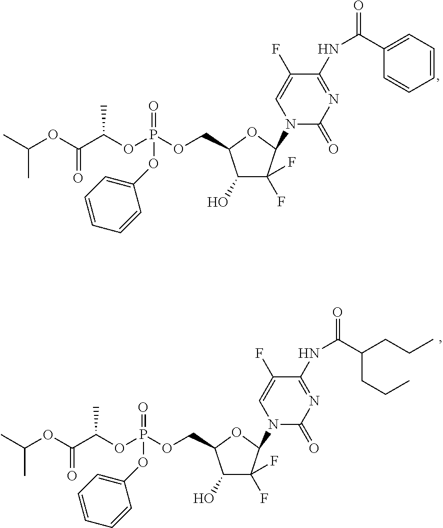 [SMILES: CC(C)OC(=O)[C@H](C)OP(=O)(OC[C@H]1O[C@@H](n2cc(F)c(NC(=O)c3ccccc3)nc2=O)C(F)(F)[C@@H]1O)Oc1ccccc1 ; CCCC(CCC)C(=O)Nc1nc(=O)n([C@@H]2O[C@H](COP(=O)(Oc3ccccc3)O[C@@H](C)C(=O)OC(C)C)[C@@H](O)C2(F)F)cc1F]
and a stereoisomer or a pharmaceutically acceptable salt thereof.

3. A pharmaceutical composition comprising a compound of claim 1 and a pharmaceutically acceptable excipient.

4. A method of treating a subject having a disease, disorder or condition comprising administering an effective amount of a compound of claim 1 to a subject in need thereof.

5. The method of claim 4, wherein the disease is selected from the group consisting of a viral disease, a cancer, liver fibrosis, fatty liver, and malaria.

6. The method of claim 5, wherein the viral disease is selected from the group consisting of hepatitis, influenza, HIV, and HPV.

7. The method of claim 5, wherein the cancer is selected from the group consisting of non-small cell lung cancer, pancreatic cancer, bladder cancer, breast cancer, liver cancer, and lymphoma.

8. (canceled)

9. (canceled)

10. (canceled)

11. A method of inhibiting or killing a cell comprising contacting the cell with an effective amount of a compound of claim 1.

12. The method of claim 11, wherein the cell is a tumor cell.

13. The method of claim 12, wherein the tumor cell is selected from the group consisting of a non-small cell lung cancer cell, a pancreatic cancer cell, a bladder cancer cell, a breast cancer cell, a liver cancer cell, and a lymphoma cell.

14. The method of claim 11, wherein the cell comprises a virus.

15. The method of claim 14, wherein the virus is selected from the group consisting of hepatitis, influenza, HIV, and HPV.

16. The method of claim 11, wherein the cell is ex vivo.

17. The method of claim 11, wherein the cell is mammalian.
